(19) United States
(12) Patent Application Publication
(10) Pub. No.: US 2019/0374445 A1
(43) Pub. Date: Dec. 12, 2019

(54) THICKENED OXIDIZING AGENT-CONTAINING FORMULATIONS IN PACKAGINGS COMPOSED OF BARRIER LAYER FILMS II

(71) Applicant: Henkel AG & Co. KGaA, Duesseldorf (DE)

(72) Inventors: Marc Nowottny, Moenchengladbach (DE); Burkhard Mueller, Duesseldorf (DE)

(73) Assignee: Henkel AG & Co. KGaA, Duesseldorf (DE)

(21) Appl. No.: 16/331,896

(22) PCT Filed: Jun. 30, 2017
(86) PCT No.: PCT/EP2017/066248

§ 371 Date: Mar. 8, 2019

(30) Foreign Application Priority Data
Sep. 9, 2016 (DE) 10 2016 217 179.8

(51) Int. Cl. A61K 8/02; A61K 8/73; A61K 8/86; A61Q 5/10; A61K 8/26; A61K 8/25; A61K 8/29; A61K 8/28; A61K 8/19

(52) U.S. Cl. CPC A61K 8/0233; A61K 8/73; A61K 8/86; A61Q 5/10; A61K 8/19; A61K 8/25; A61K 8/29; A61K 8/28; A61K 8/26

(57) ABSTRACT

The present disclosure relates to a cosmetic product for changing the natural color of keratin fibres, in particular human hair, which contains at least one packaging (VP) and a cosmetic composition (KM) found in said packaging (VP). The packaging is made of a multi-layered film (F) which contains at least two polymer layers (P1) and (P2) and at least one barrier layer (BS). The cosmetic composition comprises at least one oxidizing agent and at least on thickener from the group of xanthans. The use of the packaging (VP) in combination with the cosmetic composition (KM) surprisingly does not lead to an expansion of the packaging or an excessive loss of water from the agent (KM) during storage.

### CROSS-REFERENCE TO RELATED APPLICATION

This application is a U.S. National-Stage entry under 35 U.S.C. § 371 based on International Application No. PCT/EP2017/066248, filed Jun. 30, 2017, which was published under PCT Article 21(2) and which claims priority to German Application No. 10 2016 217 179.8, filed Sep. 9, 2016, which are all hereby incorporated in their entirety by reference.

### TECHNICAL FIELD

The present disclosure lies in the field of cosmetics and relates to a product for oxidatively changing the color of keratin fibres, in particular human hair, which product comprises an oxidizing agent-containing composition packaged in a packaging. The oxidizing agent-containing composition contains a thickener from the group of xanthans. The packaging is a packaging produced from a specific multi-layer film composite system, the wall of said packaging comprising at least two polymer layers and a barrier layer. Here, the barrier layer has a penetration barrier effect for gases and water vapour.

### BACKGROUND

The changing of the color of keratin fibres, in particular hair, represents a key area of modern cosmetics. The appearance of hair can hereby be adapted to current fashion trends and to the individual wishes of an individual person. A person skilled in the art is aware of various possibilities for changing the color of hair.

Hair color can be changed temporarily by the use of substantive dyes. Here, dyes that have already fully formed diffuse from the coloring agent into the hair fibres. Coloring by employing substantive dyes is associated with a low level of hair damage, however one disadvantage is constituted by the low durability and the quick rate at which the coloring obtained by substantive dyes can be washed out.

If the consumer desires a long-lasting color result or a color tint that is lighter than the starting hair color, oxidative color-changing agents are therefore usually used. What are known as oxidation dyes are used for permanent, intense colorations having appropriate fastness properties. Such coloring agents usually comprise oxidation dye precursors, so-called developer components and coupler components, which create the actual dyes among each other under the influence of oxidizing agents, generally hydrogen peroxide. Oxidation dyes are exemplified by excellent long-lasting dyeing results.

If hair is to be merely lightened or bleached, this is often achieved by use of oxidizing agents without addition of oxidiation dye precursors. For a medium bleaching effect, the use of hydrogen peroxide as sole oxidizing agent is sufficient, however a mixture of hydrogen peroxide and peroxydisulfate salts is usually used in order to attain a stronger bleaching effect.

Oxidative color-changing agents usually come onto the market in the form of two-component agents in which two different preparations are present packaged separately in two separate packagings and are mixed with one another only just before use.

The first preparation is a formulation—generally set to an acidic pH for reasons of stability—which as oxidizing agent contains for example hydrogen peroxide in concentrations of from about 1.5 to about 12% by weight. The oxidizing agent formulation is usually present in the form of an emulsion or dispersion and is generally provided in a plastic bottle with reclosable outlet opening (developer bottle).

This oxidizing agent formulation is mixed with a second preparation prior to use. This second preparation is a formulation set to an alkaline pH, which is often provided in the form of a cream or a gel, provided a change in color is also desired alongside the lightening effect, and additionally also contains at least one oxidation dye precursor. This second preparation can be provided for example in the form of a tube or in the form of a plastic or glass container.

In the previously described usual application form, the second preparation containing the alkalizing agent and/or the oxidation dye precursors is transferred from the tube or the container into the developer bottle and is then mixed by shaking with the hydrogen peroxide preparation already disposed in the developer bottle. The mixture for application is produced in this way in the developer bottle. The mixture is then applied to the hair via a small nozzle or outlet opening at the head of the developer bottle. The nozzle or outlet opening is opened after the shaking, and the mixture for application can be dispensed by squeezing the flexible developer bottle.

The use of the developer bottle requires a certain routine on the part of the user, and therefore some users will prefer to make the mixture for application in a mixing dish and to apply it using a brush.

When producing the mixture for application in a dish, both components—the first preparation, which contains the oxidizing agent, and the second preparation with alkalizing agent and/or oxidation dye precursor—are transferred fully to a dish or a similar vessel and are stirred there, for example using a brush. The mixture for application is then removed from the mixing dish using the brush. In this form of application, the use of a bulky and costly developer bottle is not necessary, however there is still a need for economical and material-saving packaging forms for the oxidizing agent preparation.

In this regard, packagings in pouch or bag form which are generally manufactured from plastic films or also metal foils lend themselves as an economical packaging form with low material consumption.

A packaging of this kind can be produced for example by adhesive bonding or hot pressing of two plastic films arranged one above the other, wherein the adhesive bonding is provided at all edges of the films. The interior of the packaging (i.e. of the plastic bag) produced by the adhesive bonding can then be filled with the desired cosmetic preparation. The packaging can be opened by tearing open or cutting open the plastic bag.

The filling of oxidizing agent preparations into packagings of this kind, however, is associated with problems originating in the reactivity of the oxidizing agent. Oxidizing agents are highly reactive substances which—depending on the storage conditions and possibly on the presence of impurities having a destructive effect—break down in small portions, with formation of oxygen (i.e. of gas).

The developer bottles known from the prior art are generally filled only at most to half, usually merely to one third of their internal volume with the oxidizing agent composition. Developer bottles are generally made from polyethylene. The polyethylene is permeable both to water vapour and to gases; there is no, or only very low overpressure in the developer bottle. In addition, developer bottles are usually provided with stable, thick walls and a stable screw cap, and therefore the diffusion of the water vapour or the gases is reduced by the thickness of the walls and a pressure increase occurring to a small extent within the bottle has no negative effects.

In contrast hereto, bag-like packagings however are usually filled completely with the liquid preparation, and there is practically no remaining airspace left in the filled bag. In addition, packagings of this kind should be flexible, and when opened (for example torn open or cut open) there should be no uncontrolled escape of the preparation. For this reason, the creation of overpressure in the packaging should be avoided as far as possible in the case of packaging for liquid preparations.

If an oxidizing agent composition is disposed in a packaging of this kind, the gas (oxygen) created during storage may thus cause the packaging to expand. Since the edges of the packaging are usually only adhesively bonded, a strong inflation in the worst case scenario cause the packaging to burst. For these reasons, the choice of the film material from which the packaging is made is of great importance in respect of the storage of oxidizing agent-containing compositions.

Packagings that are made of pure plastic, such as polyethylene or polypropylene, are permeable both to water vapour and to gases. When storing an oxidizing agent-containing preparation in a packaging made of polyethylene or polypropylene, there is thus no expansion of the packaging. Due to the high permeability of the comparatively thin film of the packaging to water vapour, however, the water content of the preparation reduces. If the preparation is stored in the packaging for a number of weeks to months, the water loss exceeds the maximum value allowed for sufficient storage stability.

Completely airtight packagings are produced for example from plastic films that have a lamination with a metal layer, for example with an aluminum layer. These packagings are impermeable to water vapour and gases. If these packagings are filled with an oxidizing agent-containing preparation, the gas created as the oxidizing agent breaks down therefore cannot escape, and the packaging expands as described above and can burst.

### BRIEF SUMMARY

Cosmetic products for changing the natural color of keratin fibres are provided herein. In an embodiment, a cosmetic product for changing the natural color of keratin fibres includes:
(i) a packaging, including a multi-layer film which includes a first polymer layer, a second polymer layer, and a barrier layer, and
(ii) a cosmetic composition, which is packaged in the packaging and includes:
   a) an oxidizing compound, and
   b) a thickener, selected from the group of xanthans.

In another embodiment, a cosmetic product for changing the natural colour of keratinous fibres includes:
(i) a packaging, including a multi-layer film, which includes:
   a first polymer layer including polypropylene and having a layer thickness of from about 60.0 μm to about 90.0 μm,
   a second polymer layer including polyethylene terephthalate and having a layer thickness of from about 10.0 μm to about 20.0 μm, and
   a barrier layer including:
      a layer including silicon dioxide; and
      a layer including an inorganic-organic hybrid polymer;
   wherein the barrier layer has a layer thickness of from about 10.0 nm to 150 nm and is disposed between the first polymer layer and the second polymer layer, and
(ii) a cosmetic composition, which is packaged in the packaging and includes:
   a) an oxidizing compound including hydrogen peroxide and present in a total quantity of from about 1.5 to about 15% by weight, in relation to the total weight of the cosmetic composition,
   b) a thickener, selected from the group of xanthans, present in an amount of from about 0.20 to about 6.0% by weight, in relation to the total weight of the cosmetic composition;
   c) water present in a total quantity of from about 60 to about 97% by weight, in relation to the total weight of the cosmetic composition.

In another embodiment, a cosmetic product for changing the natural colour of keratinous fibres includes:
(i) a packaging, including a multi-layer film, which includes:
   a first polymer layer including polypropylene and having a layer thickness of from about 60.0 μm to about 90.0 μm,
   a second polymer layer including polyethylene terephthalate and having a layer thickness of from about 10.0 μm to about 20.0 μm, and
   a barrier layer including:
      a layer including silicon dioxide; and
      a layer including an inorganic-organic hybrid polymer;
   wherein the barrier layer has a layer thickness of from about 10.0 nm to 150 nm and is disposed between the first polymer layer and the second polymer layer, and
(ii) a cosmetic composition, which is packaged in the packaging and consists of:
   a) an oxidizing compound,
   b) a thickener selected from the group of xanthans;
   c) water present in a total quantity of from about 60 to about 97% by weight, in relation to the total weight of the cosmetic composition;
   d) an inorganic alkalizing agent; and
   e) a mixture of acids including:
      at least one of dipicolinic acid, disodium pyrophosphate, ethylenediamine tetraacetic acid, or salts thereof; and
      1-hydroxyethane-1,1-diphosphonic acid.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present application was that of packaging the oxidizing agent composition in an economical, material-saving, space-saving and reliable manner, and in particular in a manner stable under storage.

It has now surprisingly been found that oxidizing agent-containing compositions can be packaged in a manner that is stable under storage on the one hand if specific packagings are used which are made of specific film composite systems and additionally have a barrier layer. On the other hand the oxidizing agent preparation must be thickened with specific thickeners.

The subject of the present disclosure is a cosmetic product for changing the natural color of keratin fibres, in particular human hair, comprising
(i) at least one packaging (VP), comprising at least one multi-layer film (F) which contains at least one first polymer layer (P1), at least one second polymer layer (P2), and at least one barrier layer (BS), and
(ii) at least one cosmetic composition (KM), which is packaged in the packaging (VP) and contains:
   a) at least one oxidizing compound, and
   b) at least one thickener, selected from the group of xanthans.

Keratin fibres, keratin-containing fibres or keratinous fibres are understood to mean fur, wool, feathers and in particular human hair. Although the agents as contemplated herein are suitable primarily for lightening and dyeing keratin fibres, there is in principle also nothing against use in other areas.

The product as contemplated herein is a product for oxidatively changing the color of keratin fibres, i.e. a product which is applied to the human head in order to attain an oxidative coloring, lightening, bleaching, fading or tinting of the hair. In this context, “tinting” is understood to mean coloring in which the color result is lighter than the starting hair color.

The term “packaging” is also understood within the context of the present disclosure to mean a packaging which is preferably provided in the form of a sachet. A sachet is a small packaging in pouch or bag form, which is often used in the packaging of cosmetics. The holding capacity of the packaging, in particular the sachet, can be for example from about 5 to about 1000 ml, preferably from about 10 to about 200 ml, and particularly preferably from about 20 to about 50 ml.

In addition, a multi-layer film (F) within the scope of the present disclosure is understood to mean a thin, planar web that can be rolled up and which is formed of the at least one polymer layer (P1) and the at least one polymer layer (P2). This multi-layer film (F) forms the wall of the packaging (VP). The polymer layers (P1) and (P2) preferably comprise polymers that are able to form films. Furthermore, the polymer layers (P1) and (P2) are preferably polymer layers different from one another. The packaging additionally contains a barrier layer (BS) which prevents or reduces the passage of water vapour and other gases, such as oxygen, that is to say prevents or reduces the diffusion of these gases through the wall of the packaging.

Furthermore, the term “thickener” within the scope of the present disclosure is understood to mean compounds which can bind liquids, in particular water, and which increase the viscosity of these liquids. Thickeners within the scope of the present disclosure also include gel formers, which are able to thicken liquids so as to form compositions having a gel-like consistency, or so as to form gels. Gel-like cosmetic agents or gels are understood as contemplated herein to mean dimensionally stable, easily deformable, disperse systems formed from at least two components: the gel former (usually a solid, colloid-split substance with long or heavily branched compounds) and a liquid (usually water) as dispersant. The gel former in the liquid forms a spatial network, wherein the individual gel-forming compounds adhere to one another by primary and/or secondary valences at different spatial points among one another.

The term “xanthans” is understood as contemplated herein to mean naturally occurring polysaccharides which can be obtained with the aid of bacteria of the Xanthomonas genus from sugar-containing substrates. The xanthan used as contemplated herein preferably contains d-glucose, d-mannose, d-glucuronic acid, acetate and pyruvate in a molar ratio of 28:30:20:17:5.1-6.3, wherein the main chain includes β-1,4-bound glucose units (also referred to as a cellulose chain). The xanthans used particularly preferably within the scope of the present disclosure have the CAS no. 11138-66-2 and the following structural formula:

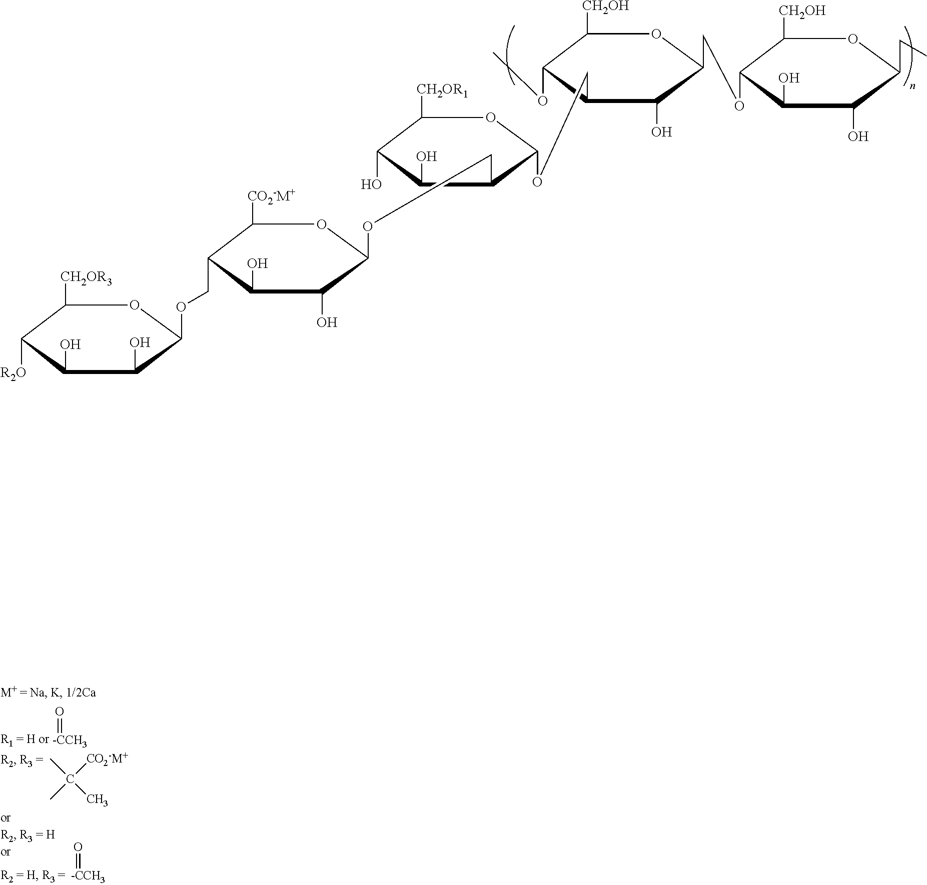

The cosmetic product as contemplated herein comprises, as first constituent, a packaging (VP) which comprises a multi-layer film (F). This film contains at least one first polymer layer (P1), at least one second polymer layer (P2), and at least one barrier layer (BS). This multi-layer film constitutes the wall or the outer casing of the packaging. As previously described, a packaging of this kind is usually produced by adhesive bonding, pressing or welding of two film pieces arranged one above the other (wherein the packaging (VP) at the same time is filled with the cosmetic composition (KM)), i.e. a packaging of this kind is closed on all sides. This packaging can be opened for example by being torn open or cut open.

The thickness of the multi-layer film (F) in this case should be such that a sufficient mechanical stability is provided, but at the same time the film (F)—and thus the packaging (VP) produced from the film—is flexible enough that complete removal of the cosmetic composition (KM) from the opened packaging (VP) is possible by a compressing or squeezing of the packaging. These requirements are met in particular if the film (F) has a certain overall thickness. Preferred embodiments of the present disclosure are therefore exemplified in that the at least one multi-layer film has a total thickness of from about 21 μm to about 2.0 mm, preferably from about 30 μm to about 1.0 mm, preferably from about 50 μm to about 500 μm, in particular from about 60 μm to about 200 μm. The total thickness of the film (F) within the sense of the present disclosure is understood to mean the sum of the thicknesses of all individual layers from which the film (F) is made.

The arrangement of the layers (P1), (P2) and (BS) within the multi-layer film (F) can be different. It is furthermore also possible that the film (F) also comprises further layers in addition to the above-mentioned layers. In addition, it is advantageous as contemplated herein if all aforementioned layers are each oriented parallel to the surfaces of the film (F), that is to say all layers have the same orientation.

It is particularly preferred as contemplated herein if the barrier layer (BS) is arranged on the side coming into contact with the cosmetic composition (KM). The first polymer layer (P1) thus on the one hand borders the barrier layer (BS) and on the other hand the second polymer layer (P2), which is disposed on the outer side of the packaging. The polymer layer (P1) is different here from the polymer layer (P2). Here, the barrier layer (BS) is applied as carrier layer to the first polymer layer (P1). The second polymer layer (P2) is then applied to the aforementioned polymer layer (P1). The three layers (BS), (P1) and (P2) together form a film (F), the total thickness of which is preferably 30 μm to 1.0 mm.

Within the scope of the present disclosure, however, an arrangement in which the barrier layer (BS) lies between the first polymer layer (P1) and the second polymer layer (P2) is particularly preferred. In this case the multi-layer film (F) includes three layers, wherein the layer (P1) is innermost and is in contact with the cosmetic composition (KM). The layer (P1) is in contact with the barrier layer (BS), and the barrier layer (BS) in turn is in contact with the layer (P2). With this layered arrangement the layers (P1) and (P2) are not adjacent to one another, but are separated by the barrier layer (BS). With this arrangement the layers (P1) and (P2) can include, in principle, the same polymer material, however it is preferred if the two layers (P1) and (P2) include different polymer materials. The three layers (P1), (BS) and (P2) together form a film (F), the total thickness of which is preferably 30 μm to 1.0 mm. The particular advantage of this arrangement lies in the fact that the—often very thin—barrier layer (BS) is disposed neither on the inner nor on the outer surface of the multi-layer film (F), but instead is protected in the direction of the inner side by the polymer layer (P1) and in the direction of the outer side by the polymer layer (P2). With this arrangement, mechanical abrasion or mechanical destruction of the barrier layer (BS) can thus be avoided to the best possible extent. It is therefore advantageous within the scope of the present disclosure if the at least one multi-layer film (F) contains the at least one barrier layer (BS) between the at least one first polymer layer (P1) and the at least one second polymer layer (P2). The use of packagings of this kind has proven to be particularly advantageous in respect of the increased storage stability, since this arrangement demonstrates no expansion or delamination in the event of relatively long periods of contact with an oxidizing agent-containing composition.

Likewise particularly preferred as contemplated herein is a film (F) in which the first polymer layer (P1) is arranged on the side coming into contact with the cosmetic composition (KM). The second polymer layer (P2) borders the polymer layer (P1) and is different therefrom. The barrier layer (BS) is disposed externally. In films (F) having this layer arrangement, the layer (P1) for example can function as a polymer carrier layer, to which the second polymer layer (P2) is then applied. The side bordering (P2) (i.e. the outer side) is then provided with the barrier layer. It is therefore advantageous within the scope of the present disclosure if the at least one multi-layer film (F) contains the at least one barrier layer (BS) on the outer side of the packaging (VP). As contemplated herein the outer side of the packaging (VP) is understood to mean the side of the packaging that does not come into contact with the cosmetic composition (KM), but instead is in contact with the surrounding environment. The three layers (P1), (P2) and (BS) here form a film (F), the total thickness of which is preferably from about 30 μm to about 1.0 mm. The use of packagings of this kind has proven to be particularly advantageous in respect of the increased storage stability, since this arrangement demonstrates no expansion or delamination in the event of relatively long periods of contact with an oxidizing agent-containing composition.

If the multi-layer film (F) contains the previously described three layers (P1), (P2) and (BS), suitable arrangements as contemplated herein of the layers are described hereinafter (as considered from the interior (in contact with the cosmetic composition (KM)) to the outer side):
a) *interior*-layer (P1)-layer (P2)-barrier layer (BS)-*outer side*,
b) *interior*-layer (P1)-barrier layer (BS)-layer (P2)-*outer side*,
c) *interior*-layer (P2)-layer (P1)-barrier layer (BS)-*outer side*,
d) *interior*-layer (P2)-barrier layer (BS)-layer (P1)-*outer side*,
e) *interior*-barrier layer (BS)-layer (P1)-layer (P2)-*outer side*,
f) *interior*-barrier layer (BS)-layer (P2)-layer (P1)-*outer side*,

This first polymer material of the first layer (P1) is an organic polymer material as contemplated herein. This material can be a layer formed of one polymer type or a layer formed of a polymer blend. This first layer (P1) can function for example as a polymer carrier material, i.e. when producing the film a layer or a film formed of the polymer material (P1) can be provided and then sprayed, laminated or coated with the further layers as contemplated herein. Preferred embodiments of the present disclosure are exemplified in that the at least one first polymer layer (P1) is formed from polypropylene, polyethylene, polyester, polyamide or polyvinyl alcohol, in particular from polypropylene. The term “is formed” is understood as contemplated herein to mean that the polymer layer contains at least about 70% by weight, preferably at least about 80% by weight, preferably at least about 90% by weight, in particular at least about 99% by weight, in each case in relation to the total weight of the polymer layer (P1), of the aforementioned compounds.

A particularly preferred product as contemplated herein is therefore exemplified in that the multi-layer film (F) comprises at least one first polymer layer (P1) which is formed from polypropylene. Polypropylene is alternatively also referred to as poly(l-methylene) and is a thermoplastic polymer belonging to the group of polyolefins. Polypropylene is produced by polymerization of propylene (propene) with use of various catalysts. Polypropylene for example can thus be produced by stereospecific polymerization of propylene in the gas phase or in suspension as described by Giulio Natta. Polypropylenes as contemplated herein can be isotactic and thus highly crystalline, or syndiotactic or amorphous. The mean relative molar mass can be controlled for example by setting a certain hydrogen partial pressure during the polymerization of the propene. For example, polypropylene can have mean relative molar masses of approximately 150,000 to 1,500,000 g/mol. Polypropylene can be processed for example by extrusion and stretch blow moulding methods, or by pressing, calendering, thermoforming and cold-forming.

The first polymer layer (P1) preferably has a certain layer thickness. It is therefore preferred within the scope of the present disclosure if the at least one first polymer layer (P1) has a layer thickness of from about 20.0 μm to about 300 μm, preferably from about 40.0 μm to about 200 μm, preferably from about 50.0 μm to about 100 μm, in particular from about 60.0 μm to about 90.0 μm.

A particularly preferred product as contemplated herein is therefore exemplified in that the multi-layer film (F) comprises at least one first polymer layer (P1) which is formed from polypropylene and has a layer thickness of from about 60.0 to about 90.0 μm.

Furthermore, the multi-layer film (F) from which the packaging is produced comprises a second polymer layer (P2) made of a second polymer material. This material can be a layer formed of one polymer type or a layer formed of a polymer blend. When producing the multi-layer film, the second layer (P2) for example—either before or after application of the barrier layer (BS) to the first polymer layer (P1) functioning as carrier layer, can be sprayed on, spread on or stratified. However, it is also conceivable that the second polymer layer (P2) functions as carrier layer, to which the barrier layer (BS) and the first polymer layer (P1) are then applied.

Depending on the previously described sequence of the coating, the first polymer material of the first polymer layer (P1) and the second polymer material of the second polymer layer (P2) can be either the same (if the two layers are not in contact with one another) or also different. The polymer layer (P2) can therefore be formed from the compounds mentioned previously in conjunction with the polymer layer (P1). The layers (P1) and (P2) are preferably produced from different polymer materials (i.e. different polymers or polymer blends). It is therefore preferred within the scope of the present disclosure if the at least one second polymer layer (P2) is formed from polyethylene terephthalate or polyethylene naphthalate, in particular from polyethylene terephthalate. The term “is formed” is understood as contemplated herein to mean that the polymer layer contains at least about 70% by weight, preferably at least about 80% by weight, preferably at least about 90% by weight, in particular at least about 99% by weight, in each case in relation to the total weight of the polymer layer (P2), of the aforementioned compounds. Polyethylene terephthalate (PET) is a polymer from the group of polyesters. Polyethylene terephthalate can be produced for example by transesterification of dimethyl terephthalate with ethylene glycol at higher temperatures. In this transesterification reaction, methanol is cleaved off and is removed by distillation. The resultant bis(2-hydroxyethyl)terephthalate is reacted by polycondensation to form PET, wherein ethylene gycol is created in turn. A further method for producing polyethylene terephthalate is direct polycondensation of ethylene glycol and terephthalic acid at high temperatures with the resultant water being distilled off.

The second polymer layer (P2) preferably has a smaller layer thickness than the polymer layer (P1). It is therefore advantageous within the scope of the present disclosure if the at least one second polymer layer (P2) has a layer thickness of from about 1.00 μm to about 100 μm, preferably from about 2.50 μm to about 50.0 μm, preferably from about 5.00 μm to about 25.0 μm, in particular from about 10.0 μm to about 20.0 μm.

A particularly preferred product as contemplated herein is therefore exemplified in that the multi-layer film (F) comprises at least one second polymer layer (P2) which is formed from polyethylene terephthalate and has a layer thickness of from about 10.0 to about 20.0 μm.

The polymer layers (P1) and (P2) of the multi-layer film (F) include organic polymer materials which generally have only an insufficient barrier effect with respect to gases and water vapour. If the oxidizing agent-containing composition (KM) is packaged in a packaging (VP) formed from a multi-layer film (F) which comprises merely the two organic polymer layers (P1) and (P2), water vapour can escape unhindered, and therefore the water content in the composition (KM) changes unacceptably when said composition is stored over a relatively long period. In order to selectively minimize the uncontrolled escape of water vapour from the packaging (VP), the organic polymer layers (P1) and (P2) are therefore used in combination with a barrier layer (BS).

The barrier layer (BS) has a penetration barrier effect for gases and water vapour. What is meant by this as contemplated herein is that the barrier layer (BS) reduces the rate of permeation of water vapour and of gases through the film. A film (F) as contemplated herein which also has a barrier layer (BS) in addition to the layers (P1) and (P2) thus has a reduced water vapour permeability and a reduced gas permeability compared to a comparable film (of equal total thickness) which comprises merely the two layers (P1) and (P2), but no barrier layer (BS).

The barrier layer (BS) for example is a thin layer which comprises inorganic material, wherein the inorganic material can be applied to the organic polymer layer (P1) and/or (P2) with the aid of vacuum coating techniques (for example PVD or physical vapour deposition, or CVD or chemical vapour deposition).

If the barrier layer (BS) is a layer which comprises at least one inorganic material, for example aluminum, aluminum oxides, magnesium, magnesium oxides, silicon, silicon oxides, titanium, titanium oxides, tin, tin oxides, zirconium, zirconium oxide and/or carbon can be considered here. In this regard, oxides are particularly preferably used and can be selected from the group of aluminum oxides, magnesium oxides, silicon oxides, titanium oxides, tin oxides and/or zirconium oxides. The barrier layer (BS) formed from inorganic material is located very particularly preferably between the two polymer layers (P1) and (P2). The production of films with barrier layers formed from inorganic material is described for example in document EP 1036813 A1, the entire content of which is referred to at this juncture.

The barrier layer (BS) can also comprise a thin layer formed from inorganic-organic hybrid polymers. These polymers are known in the literature under the specialist term ORMOCER polymers. A typical ORMOCER polymer can be produced for example with hydrolytic polycondensation of an organofunctional silane with an aluminum compound and optionally with an inorganic oxide component. Appropriate syntheses are disclosed for example in document EP 0792846 B 1, the entire content of which is referred to at this juncture. Inorganic-organic hybrid polymers (ORMOCER polymers) comprise both inorganic and organic network structures. The inorganic siliceous network structure can be constructed in a sol-gel process via the controlled hydrolysis and condensation of alkoxysilanes. By additionally incorporating metal alkoxides in the sol-gel process, the siliceous network can be modified selectively. By polymerization of organofunctional groups which are introduced into the material by the organoalkoxylanes, an organic network is additionally constructed. The ORMOCER polymers produced in this way can be applied for example by employing conventional application techniques (spraying, spreading, etc.) to the layers (P1) and/or (P2).

Preferred embodiments of the present disclosure are therefore exemplified in that the at least one barrier layer (BS) is formed from aluminum oxides, magnesium oxides, silicon oxides, titanium oxides, tin oxides, zirconium oxides, inorganic-organic hybrid polymers (ORMOCER polymers) or mixtures thereof, in particular from silicon oxides. The term “is formed” is understood as contemplated herein to mean that the polymer layer contains at least about 70% by weight, preferably at least about 80% by weight, preferably at least about 90% by weight, in particular at least about 99% by weight, in each case in relation to the total weight of the barrier layer (BS), of the aforementioned compounds. Particularly preferred are multi-layer films (F) as contemplated herein in which the barrier layer (BS) is formed from silicon oxides or inorganic-organic hybrid polymers (ORMORCER polymers).

It is furthermore likewise possible that the multi-layer film (F), which constitutes the wall of the packaging (VP), has a barrier layer (BS) which comprises both inorganic oxide components and inorganic-organic hybrid polymers (ORMOCER polymers). In addition, the barrier layer (BS) may also comprise a further organic polymer material which itself has no barrier effect, but for example increases the mechanical stability of the barrier layer, simplifies the production, or brings about improved adhesive bonding of the layers (BS) and (P1) and/or (P2). Particularly preferred are multi-layer films (F) as contemplated herein in which the barrier layer (BS) is formed from aluminum oxides, magnesium oxides, silicon oxides, titanium oxides, tin oxides, zirconium oxides and mixtures thereof and additionally at least one inorganic-organic hybrid polymer (ORMORCER polymers).

The thicker is the barrier layer (BS), the greater or stronger is the permeation barrier effect for gases and water vapour. The thickness of the barrier layer (BS) can therefore be selected depending on the desired barrier layer effect. The barrier layer (BS) can have a layer thickness of from about 1 to about 1000 nm (nanometers), for example. The barrier layer (BS) preferably has a layer thickness of from about 5 to about 500 nm, more preferably from about 10 to about 250 nm, and particularly preferably from about 10 to about 150 nm (nanometers). Preferred embodiments of the present disclosure are therefore exemplified in that the at least one barrier layer (BS) has a layer thickness of from about 1.00 nm to about 1000 nm, preferably from about 5.00 nm to about 500 nm, preferably from about 10.0 nm to about 250 nm, in particular from about 10.0 nm to about 150 nm.

Besides the previously described layers (P1), (P2) and (BS), the multi-layer film (F) may additionally also comprise one or more further layers. These further layers can be intermediate layers and/or adhesive layers, for example. It is therefore preferred as contemplated herein if the at least one multi-layer film (F) additionally contains at least one further layer, selected from the group of intermediate layers (SZ), adhesive layers (SK) and mixtures thereof.

For example, the films (F) can have further intermediate layers (SZ) in order to increase the mechanical stability. Intermediate layers can also prevent or minimize the permeation of polymers or remaining monomers from a polymer layer into the cosmetic composition (KM).

In order to increase the bond strength, the films can additionally also comprise one or more adhesive layers (SK) so as to reduce or to prevent a delamination (i.e. a detachment or the formation of an air pocket) between two layers.

A particularly preferred product as contemplated herein is exemplified in that the multi-layer film (F), besides the first polymer layer (P1), the second polymer layer (P2) and the barrier layer (BS), additionally also contains one or more further layers which are selected from intermediate layers (SZ) and/or adhesive layers (SK).

If the multi-layer film (F), in addition to the layers (P1), (P2) and (BS), also contains further layers, suitable arrangements as contemplated herein of the layers are described hereinafter (as considered from the interior (in contact with the cosmetic composition (KM)) to the outer side):
a) *interior*-layer (P1)-first adhesive layer (SK1)-layer (P2)-second adhesive layer (SK2)-barrier layer (BS)-*outer side*,
b) *interior*-layer (P1)-adhesive layer (SK1)-layer (P2)-barrier layer (BS)-*outer side*,
c) *interior*-layer (P1)-layer (P2)-second adhesive layer (SK2)-barrier layer (BS)-*outer side*,
d) *interior*-barrier layer (BS)-first adhesive layer (SK1)-layer (P1)-second adhesive layer (SK2)-layer (P2)-*outer side*,
e) *interior*-barrier layer (BS)-adhesive layer (SK)-layer (P1)-layer (P2)-*outer side*,
f) *interior*-barrier layer (BS)-layer (Si)-adhesive layer (SK)-layer (P2)-*outer side*,
g) *interior*-layer (P1)-first adhesive layer (SK1)-barrier layer (BS)-second adhesive layer (SK2)-layer (P2)-*outer side*,
h) *interior*-layer (P1)-adhesive layer (SK)-barrier layer (BS)-layer (P2)-*outer side*,
i) *interior*-layer (P1)-barrier layer (BS)-adhesive layer (SK)-layer (P2)-*outer side*,

The product as contemplated herein, as second constituent, comprises a cosmetic composition (KM) which is packaged in the packaging (VP) and comprises at least one oxidizing agent, and a thickener from the group of xanthans.

The purpose of the product as contemplated herein is to provide an oxidative color change. To this end—as already described previously—a cosmetic composition (KM) containing an oxidizing agent is usually mixed with a second preparation (B) packaged separately from (KM). In this way, the ready-to-use oxidative color-changing agent is produced. The preparation (B) can contain different ingredients depending on whether a bleaching, lightening or coloring is to be attained by the oxidative color change. If merely lightening or bleaching is to be attained, the preparation (B) contains at least one alkalizing agent. If oxidative coloring is desired, oxidation dye precursors are thus often also contained in the preparation (B) in addition to the alkalizing agent. In order to ensure sufficiently quick miscibility of the preparations (KM) and (B), both the preparation (KM) and the preparation (B) are usually flowable, aqueous or water-containing preparations.

The preparation (KM) is an aqueous preparation as contemplated herein. The water content of the preparation (KM) can lie—in relation to the total weight of the preparation (KM)—for example at from about 60 to about 97% by weight, preferably at from about 75 to about 93% by weight, preferably at from about 78 to about 91% by weight, in particular at from about 80 to about 88.0% by weight. All weight values in % by weight relate here to the total weight of water contained in the preparation (KM), this being set in relation to the total weight of the preparation (KM).

The cosmetic composition (KM) contains at least one oxidizing agent as first essential ingredient a). Certain oxidizing agents are preferably used here. It is therefore advantageous within the scope of the present disclosure if the cosmetic composition (KM) contains at least one oxidizing compound selected from the group of persulfates, chlorites, hydrogen peroxide and addition products of hydrogen peroxide with urea, melamine and sodium borate, in particular hydrogen peroxide. The use of hydrogen peroxide has proven to be particularly advantageous as contemplated herein.

The concentration of the oxidizing agent in the composition (KM) is determined on the one hand by the legal specifications and on the other hand by the desired effect; from about 0.5 to about 20.0% by weight solutions in water are preferably used. It is therefore preferred as contemplated herein if the cosmetic composition (KM) contains the at least one oxidizing compound, in particular hydrogen peroxide, in a total amount of from about 0.1 to about 10% by weight, preferably from about 0.2 to about 8.0% by weight, preferably from about 0.2 to about 7.0% by weight, in particular from about 0.2 to about 6.0% by weight, in relation to the total weight of the cosmetic composition (KM). The higher is the content of oxidizing agent, in particular hydrogen peroxide, in the composition (KM), the greater is the amount of gas created with partial breakdown of the oxidizing agent. Oxidizing agent-containing preparations of higher concentration are therefore much more difficult to package in a packaging (VP) so as to be stable under storage than preparations of lower concentration. The amount of hydrogen peroxide relates here to about 100% hydrogen peroxide.

During the course of works leading to this present disclosure, it was found that the product as contemplated herein was in particular also suitable for the packaging and stable storage of hydrogen peroxide preparations (KM) of higher concentration. Packagings (VP) as contemplated herein that contained preparations (KM) with from about 9 to about 12% by weight hydrogen peroxide did not demonstrate any changes in volume (i.e. no expansion) or any unplanned openings (i.e. the packagings did not burst open), even when stored over a number of weeks at increased temperature.

The cosmetic composition (KM) contains at least one thickener from the group of xanthans as second essential ingredient b). The use of xanthans has proven to be particularly advantageous since these ensure a sufficient thickening over a long period of time even under highly acidic conditions and in the presence of an oxidizing agent. The thickening ensures that the cosmetic composition (KM) can be easily handled. In order to ensure sufficient thickening, the at least one thickener is used in certain amount ranges. It is therefore advantageous within the scope of the present disclosure if the cosmetic composition (KM) contains the at least one thickener selected from the group of xanthans in a total amount of from about 0.1 to about 10% by weight, preferably from about 0.2 to about 8.0% by weight, preferably from about 0.2 to about 7.0% by weight, in particular from about 0.2 to about 6.0% by weight, in relation to the total weight of the cosmetic composition (KM). Within the scope of the present disclosure the use of xanthans which have a mean particle diameter D₅₀ of from about 140 to about 200 μm and a viscosity (0.3% by weight solution in 0.3% KCl) of from about 250 to about 800 mPas (measured with a Brookfield viscometer at 3 rpm) has proven to be expedient. Xanthans of this kind are obtainable for example under the trade name Keltrol CG-SFT from the company CP Kelco.

During the course of the works performed for this present disclosure it was found that the use of the aforementioned specific thickener ensures that the cosmetic composition (KM), which contains at least one oxidizing agent, can be packaged and stored in the specific packaging (VP) without this packaging—which has a barrier layer with a penetration barrier effect for gases and water vapour—expanding or bursting.

In this regard, a very specific combination of oxidizing agent and thickener has proven to be advantageous. In a preferred embodiment the product as contemplated herein is therefore exemplified in that the cosmetic composition (KM) contains hydrogen peroxide and xanthans.

For further optimization of the storage stability, the aforementioned compounds are preferably used in certain amount ranges in the preparation (KM). Particularly preferred embodiments are therefore exemplified in that the cosmetic composition (KM) contains
a) from about 1.5 to about 15% by weight of hydrogen peroxide, and
b) from about 0.2 to about 6.0% by weight of xanthan, in each case in relation to the total weight of the cosmetic agent (KM).

The cosmetic composition (KM) preferably has an acidic pH value so as to avoid or reduce breakdown of the used oxidizing agent, in particular the hydrogen peroxide. It is therefore preferred within the scope of the present disclosure if the cosmetic compositions (KM) have a pH value (measured at 20° C.) of from about pH 1.5 to about pH 5.0, preferably from about pH 2.0 to about pH 4.6, preferably from about pH 2.3 to about pH 4.5, in particular from about pH 2.5 to about pH 4.0.

The preparation (KM) disposed in the packaging (VP) contains the essential ingredients in an aqueous or aqueous-alcoholic carrier, which for example can be a cream, an emulsion, a gel or also a surfactant-containing foaming solution. In order to set the desired properties of these administration forms the preparation (KM) may also contain additional active substances, auxiliaries and additives.

The preparation (KM) can for example also additionally contain one or more acids for stabilizing the used oxidizing agent, in particular the hydrogen peroxide. It is therefore preferred within the scope of the present disclosure if the cosmetic composition (KM) additionally contains at least one acid selected from the group of dipicolinic acid, citric acid, acetic acid, malic acid, lactic acid, tartaric acid, hydrochloric acid, phosphoric acid, pyrophosphoric acid and salts thereof, benzoic acid and salts thereof, 1-hydroxyethane-1-1-diphosphonic acid, ethylenediaminetetracetic acid and salts thereof, sulfuric acid and mixtures thereof, in particular a mixture of dipicolinic acid, disodium pyrophosphate, ethylenediaminetetraacetic acid and salts thereof and 1-hydroxyethane-1,1-diphosphonic acid.

A particularly high stabilization of the oxidizing agent, in particular the hydrogen peroxide, is achieved if the aforementioned acids are used in certain amount ranges. It is therefore advantageous in this context if the at least one acid, in particular the mixture of dipicolinic acid, disodium pyrophosphate, ethylenediaminetetraacetic acid and salts thereof and 1-hydroxyethane-1,1-diphosphonic acid, is contained in a total amount of from about 0.1 to about 3.0% by weight, preferably from about 0.5 to about 2.5% by weight, preferably from about 0.8 to about 2.0% by weight, in particular from about 0.9 to about 1.5% by weight, in relation to the total weight of the cosmetic composition (KM).

Particularly preferred embodiments AF 1 to AF 16 of the cosmetic composition (KM) contained in the packaging (VP) are specified in the tables below (all values in % by weight unless stated otherwise).

| | AF 1 | AF 2 | AF 3 | AF 4 |
|---|---|---|---|---|
| oxidizing agent | 0.5-20 | 1.0-18 | 1.2-16 | 1.5-15 |
| xanthan | 0.1-10 | 0.2-8.0 | 0.2-7.0 | 0.2-6.0 |
| cosmetic carrier ¹⁾ | to 100 | to 100 | to 100 | to 100 |
| | AF 5 | AF 6 | AF 7 | AF 8 |
| oxidizing agent ⁴⁾ | 0.5-20 | 1.0-18 | 1.2-16 | 1.5-15 |
| xanthan | 0.1-10 | 0.2-8.0 | 0.2-7.0 | 0.2-6.0 |
| cosmetic carrier ¹⁾ | to 100 | to 100 | to 100 | to 100 |
| | AF 9 | AF 10 | AF 11 | AF 12 |
| oxidizing agent ⁴⁾ | 0.5-20 | 1.0-18 | 1.2-16 | 1.5-15 |
| xanthan | 0.1-10 | 0.2-8.0 | 0.2-7.0 | 0.2-6.0 |
| acid | 0.1-3.0 | 0.50-2.5 | 0.8-2.0 | 0.9-1.5 |
| cosmetic carrier ¹⁾ | to 100 | to 100 | to 100 | to 100 |
| | AF 13 | AF 14 | AF 15 | AF 16 |
| oxidizing agent ⁴⁾ | 0.5-20 | 1.0-18 | 1.2-16 | 1.5-15 |
| xanthan | 0.1-10 | 0.2-8.0 | 0.2-7.0 | 0.2-6.0 |
| acid | 0.1-3.0 | 0.50-2.5 | 0.8-2.0 | 0.9-1.5 |
| cosmetic carrier ¹⁾ | to 100 | to 100 | to 100 | to 100 |

¹⁾ aqueous or aqueous-alcoholic carrier
²⁾ hydrogen peroxide, amount calculated on the basis of 100% hydrogen peroxide,
³⁾ selected from a mixture of dipicolinic acid, disodium pyrophosphate and 1-hydroxyethane-1,1-diphosphonic acid.

The previously described embodiments AF 1 to 16 are each packaged in packagings (VP) which comprise the arrangement described hereinafter of the multi-layer film (F) (from the interior (in contact with the cosmetic composition (KM)) to the outer side):
a) *interior*-layer (P1)-layer (P2)-barrier layer (BS)-*outer side*,
b) *interior*-layer (P1)-barrier layer (BS)-layer (P2)-*outer side*,
c) *interior*-layer (P2)-layer (P1)-barrier layer (BS)-*outer side*,
d) *interior*-layer (P2)-barrier layer (BS)-layer (P1)-*outer side*,
e) *interior*-barrier layer (BS)-layer (P1)-layer (P2)-*outer side*,
f) *interior*-barrier layer (BS)-layer (P2)-layer (P1)-*outer side*,
g) *interior*-layer (P1)-first adhesive layer (SK1)-layer (P2)-second adhesive layer (SK2)-barrier layer (BS)-*outer side*,
h) *interior*-layer (P1)-adhesive layer (SK1)-layer (P2)-barrier layer (BS)-*outer side*,
i) *interior*-layer (P1)-layer (P2)-second adhesive layer (SK2)-barrier layer (BS)-*outer side*,
j) *interior*-barrier layer (BS)-first adhesive layer (SK1)-layer (P1)-second adhesive layer (SK2)-layer (P2)-*outer side*,
k) *interior*-barrier layer (BS)-adhesive layer (SK)-layer (P1)-layer (P2)-*outer side*,
l) *interior*-barrier layer (BS)-layer (Si)-adhesive layer (SK)-layer (P2)-*outer side*,
m)*interior*-layer (P1)-first adhesive layer (SK1)-barrier layer (BS)-second adhesive layer (SK2)-layer (P2)-*outer side*,
n) *interior*-layer (P1)-adhesive layer (SK)-barrier layer (BS)-layer (P2)-*outer side*,
o) *interior*-layer (P1)-barrier layer (BS)-adhesive layer (SK)-layer (P2)-*outer side*,

The products as contemplated herein obtainable in this way have a high storage stability and a sustain a water loss during storage that lies within an acceptable range. No expansion or delamination of the packaging (VP) was observed during the storage of these cosmetic products as contemplated herein.

The product as contemplated herein is used for the purpose of oxidative color changing. To this end the preparation (KM) packaged in the packaging (VP), which is the oxidizing agent preparation, is mixed with at least one further preparation (B) in order to produce the ready-for-use color-changing agent. In order to prevent incompatibilities or in order to avoid a premature reaction, the preparations (KM) and (B) are packaged separately from one another.

A particularly preferred product as contemplated herein comprises a preparation (B) packaged separately from the preparation (KM), wherein the preparation (B) contains at least one compound selected from oxidation dye precursors, substantive dyes, alkalizing agents, and mixtures thereof. Preferred products of the present disclosure additionally comprise at least one second cosmetic composition (KM2) which contains at least one compound selected from oxidation dye precursors, substantive dyes, alkalizing agents and mixtures thereof and which is packaged separately from the cosmetic composition (KM).

If oxidative coloring is desired, the preparation (B) contains at least one oxidation dye precursor. Oxidation dye precursors can be divided into developers and couplers, wherein the developers, on account of their greater sensitivity to oxygen, are usually used in the form of their physiologically acceptable salts (for example in the form of their hydrochlorides, hydrobromides, hydrogen sulfates or sulfates). Coupler components, within the scope of oxidative dyeing, do not alone form any significant coloration, but instead always require the presence of developer components. Such agents preferably contain at least one oxidation dye precursor of the developer type and at least one oxidation dye precursor of the coupler type. Particularly suitable oxidation dye precursors of the developer type are selected here from at least one compound from the group formed from p-phenylenediamine, p-toluenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo[1,2-a]pyrazol-1-one and physiologically acceptable salts thereof.

Particularly preferred oxidation dye precursors of the coupler type are selected here from the group formed from 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or mixtures of these compounds or physiologically acceptable salts thereof.

The preparation (B) can additionally also contain one or more substantive dyes. Suitable non-ionic substantive dyes can be selected from the group: HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, 1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylendiamine, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

Suitable anionic substantive dyes can be selected from the group formed from Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue and tetrabromophenol blue.

Suitable cationic substantive dyes are cationic triphenylmethane dyes, such as Basic Blue 7, Basic Blue 26, Basic Violet 2 and Basic Violet 14, aromatic systems which are substituted with a quaternary nitrogen group, such as Basic Yellow 57, Basic Red 76, Basic Blue 99, Basic Brown 16 and Basic Brown 17, cationic anthraquinone dyes, such as HC Blue 16 (Bluequat B) and substantive dyes which contain a heterocycle comprising at least one quaternary nitrogen atom, in particular Basic Yellow 87, Basic Orange 31 and Basic Red 51. The cationic substantive dyes that are sold under the trade name Arianor are likewise suitable cationic substantive dyes as contemplated herein.

Dyeing processes on keratin fibers are usually performed in an alkaline environment. In order to be as gentle as possible on the keratin fibers and also on the skin, however, the setting of an excessively high pH value is not desirable. It is therefore preferred if the pH value of the agent (B) lies between 7 and 11, in particular between 8 and 10.5, The pH values in the sense of the present disclosure are pH values that were measured at a temperature of 22° C.

The preparation (B) can contain at least one alkalizing agent. The alkalizing agent usable as contemplated herein to set the preferred pH value can be selected from the group that is formed from ammonia, alkanolamines, basic amino acids, and inorganic alkalizing agents, such as alkali or alkaline earth metal hydroxides, alkali or alkaline earth metal metasilicates, alkali or alkaline earth metal phosphates, and alkali or alkaline earth metal hydrogen phosphates. Preferred inorganic alkalizing agents are magnesium carbonate, sodium hydroxide, potassium hydroxide, sodium silicate and sodium metasilicate. Organic alkalizing agents that can be used as contemplated herein are preferably selected from monoethanolamine, 2-amino-2-methyl propanol and triethanolamine. The basic amino acids usable as alkalizing agents as contemplated herein are preferably selected from the group formed from arginine, lysine, ornithine and histidine, particularly preferably arginine. It has been found within the scope of tests performed in relation to the present disclosure that agents that are preferred as contemplated herein are also exemplified in that they additionally contain an organic alkalizing agent. One embodiment of the first subject matter of the present disclosure is exemplified in that the agent additionally contains at least one alkalizing agent which is selected from the group formed from ammonia, alkanolamines and basic amino acids, in particular from ammonia, monoethanolamine and arginine or acceptable salts thereof.

The preparation (B) can also contain additional active substances, auxiliaries and additives. For example, one or more fatty constituents formed from the group of C₁₂-C₃₀ fatty alcohols, the C₁₂-C₃₀ fatty acid triglycerides, the C₁₂-C₃₀ fatty acid monoglycerides, the C₁₂-C₃₀ fatty acid diglycerides and/or the hydrocarbons can thus be contained.

A surface-active substance can preferably additionally be added in the preparation (B), wherein such surface-active substances are referred to as surfactants or as emulsifiers depending on the field of application: They are preferably selected from anionic, zwitterionic, amphoteric and non-ionic surfactants and emulsifiers.

The preparation (B) preferably contains at least one anionic surfactant. Preferred anionic surfactants are fatty acids, alkyl sulfates, alkyl ether sulfates, and ether carboxylic acids with from about 10 to about 20 C atoms in the alkyl group and up to about 16 glycol ether groups in the molecule.

The preparation (B) can also additionally contain at least one zwitterionic surfactant. Preferred zwitterionic surfactants are betaines, N-alkyl-N,N-dimethylammonium glycinates, N-acyl-aminopropyl-N,N-dimethylammonium glycinates, and 2-alkyl-3-carboxymethyl-3-hydroxyethyl-imidazolines. A preferred zwitterionic surfactant is known under the INCI name Cocamidopropyl Betaine.

It can additionally be provided that the preparation (B) contains at least one amphoteric surfactant. Preferred amphoteric surfactants are N-alkyl glycines, N-alkyl propionic acids, N-alkylaminobutyric acids, N-alkyliminodipropionic acids, N-hydroxyethyl-N-alkylamidopropylglycines, N-alkyl taurines, N-alkyl sarcosines, 2-alkylaminopropionic acids and alkylaminoacetic acids. Particularly preferred amphoteric surfactants are N-coco-alkylaminopropionate, coco-acylaminoethylaminopropionate and C₁₂-C₁₈ acyl sarcosine.

It has also proven advantageous if the preparation (B) contains further non-ionogenic surface-active substances. Preferred non-ionic surfactants have proven to be alkylpolyglycosides and alkylene oxide addition products with fatty alcohols and fatty acids, with in each case from about 2 to about 30 mol ethylene oxide per mol fatty alcohol or fatty acid. Preparations having excellent properties are also obtained if they contain fatty acid esters of ethoxylated glycerol as non-ionic surfactants.

The non-ionic, zwitterionic or amphoteric surfactants are used in amounts of from about 0.1 to about 45% by weight, preferably from about 1 to about 30% by weight, and very particularly preferably from about 1 to about 15% by weight, in relation to the total weight of the preparation (B).

The preparation (B) can additionally also contain at least one thickener. There are in principle no limitations in respect of this thickener. Both organic and purely inorganic thickeners can be used. Suitable thickeners are anionic, synthetic polymers, cationic, synthetic polymers, naturally occurring thickeners, such as non-ionic guar gums, sclerogucan gums, or xanthan gums, gum arabic, gum ghatti, karaya gum, tragacanth gum, carrageenan gum, agar-agar, locust bean flour, pectins, alginates, starch fractions, and derivatives such as amylose, amylopectin, and dextrins, as well as cellulose derivatives such as methylcellulose, carboxyalkylcelluloses, and hydroxyalkylcelluloses, non-ionic, fully synthetic polymers such as polyvinyl alcohol or polyvinylpyrrolidinone, and inorganic thickeners, in particular sheet silicates such as bentonite, in particular smectites, such as montmorillonite or hectorite.

The preparation (B) can also contain further active substances, auxiliaries and additives, such as non-ionic polymers, such as vinylpyrrolidinone/vinyl acrylate copolymers, polyvinylpyrrolidinone, vinylpyrrolidinone/vinyl acetate copolymers, polyethylene glycols, and polysiloxanes; additional silicones such as volatile or non-volatile, straight-chain, branched or cyclic, crosslinked or noncrosslinked polyalkylsiloxanes (such as dimethicones or cyclomethicones), polyarylsiloxanes, and/or polyalkylarylsiloxanes, particularly polysiloxanes with organofunctional groups, such as substituted or unsubstituted amines (amodimethicones), carboxyl, alkoxy, and/or hydroxyl groups (dimethicone copolyols), linear polysiloxane(A)-polyoxyalkylene(B) block copolymers, grafted silicone polymers; cationic polymers such as quaternized cellulose ethers, polysiloxanes with quaternary groups, dimethyldiallylammonium chloride polymers, acrylamide-dimethyldiallylammonium chloride copolymers, dimethylaminoethylmethacrylate-vinylpyrrolidinone copolymers quaternized with diethylsulfate, vinylpyrrolidinone-imidazolinium-methochloride copolymers, and quaternized polyvinyl alcohol; zwitterionic and amphoteric polymers; anionic polymers such as polyacrylic acids or crosslinked polyacrylic acids; structurants such as glucose, maleic acid, and lactic acid, hair-conditioning compounds such as phospholipids, for example, lecithin and kephalins; perfume oils, dimethyl isosorbide, and cyclodextrins; active substances that improve the fibre structure, in particular mono-, di- and oligosaccharides such as glucose, galactose, fructose, fruit sugars and lactose; dyes for staining the product; antidandruff agents such as piroctone olamine, zinc omadine, and climbazole; amino acids and oligopeptides; protein hydrolysates with an animal and/or vegetable base, and in the form of their fatty acid condensation products or optionally anionically or cationically modified derivatives; fatty substances and vegetable oils; light stabilizers and UV blockers; active substances such as panthenol, pantothenic acid, pantolactone, allantoin, pyrrolidinone carboxylic acids, and salts thereof, as well as bisabolol; polyphenols, particularly hydroxycinnamic acids, 6,7-dihydroxycoumarins, hydroxybenzoic acids, catechins, tannins, leukoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetration agents such as glycerol, propylene glycol monoethyl ether, carbonates, hydrogen carbonates, guanidines, ureas, and primary, secondary, and tertiary phosphates; opacifiers such as latex, styrene/PVP, and styrene/acrylamide copolymers; pearlescent agents such as ethylene glycol mono- and distearate and PEG-3 distearate; and pigments.

The selection of these further substances is made by a person skilled in the art in accordance with the desired properties of preparation (B) and of the product as contemplated herein. With regard to further optional components and the used amounts of these components, reference is made expressly to the relevant handbooks known to a person skilled in the art. The additional active substances and auxiliaries are used in the preparation (B) preferably in amounts of, in each case, from about 0.0001 to about 25% by weight, in particular from about 0.0005 to about 15% by weight, in each case in relation to the total weight of the preparation (B).

The following examples explain the present disclosure, but without limiting the present disclosure thereto.

### Examples

A layer 100 nm thick of silicon dioxide SiOx was applied by vapour deposition to a film layer of polyethylene terepthalate with a thickness of 12 μm (micrometers). The SiOx layer was then coated with approximately 3 g/m2 ORMOCER polymer and cured. A layer 70 μm (micrometer) thick of polypropylene was then applied to the ORMOCER layer. A packaging (VP) was produced from the film.

The packaging (VP) was filled in each case with the following preparation (KM1) (all values in % by weight). Hydrogen peroxide is contained preferably as oxidizing agent.

| Ingredients | KM1 |
|---|---|
| sodium hydroxide (50%) | 0.66 |
| dipicolinic acid | 0.10 |
| Disodium pyrophosphate | 0.030 |
| 1-hydroxyethane-1,1-diphosphonic acid (60%) | 1.5 |
| xanthan | 2.0 |
| oxidizing agent ¹⁾ | 4.1 |
| 1,2-propanediol | 4.0 |
| water | to 100 |

The cosmetic composition KM1 was filled into each of the previously described packagings (VP). The packagings were then stored for 24 weeks at 40° C. The packagings were did not suffer any expansion or delamination.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product for changing the natural color of keratin fibres, in particular human hair, comprising
(i) at least one packaging (VP), comprising at least one multi-layer film (F) which contains at least one first polymer layer (P1), at least one second polymer layer (P2), and at least one barrier layer (BS), and
(ii) at least one cosmetic composition (KM), which is packaged in the packaging (VP) and contains:
a) at least one oxidizing compound, and
b) at least one thickener, selected from the group of xanthans.

2. The cosmetic product according to claim 1, characterized in that the at least one multi-layer film contains the at least one barrier layer (BS) between the at least one first polymer layer (P1) and the at least one second polymer layer (P2).

3. The cosmetic product according to either one of claim 1 or 2, characterized in that the at least one first polymer layer (P1) is formed from polypropylene, polyethylene, polyester, polyamide or polyvinyl alcohol, in particular from polypropylene.

4. The cosmetic product according to any one of the preceding claims, characterized in that the at least one first polymer layer (P1) has a layer thickness of from 20.0 to 300 μm, preferably from 40.0 μm to 200 μm, preferably from 50.0 μm to 100 μm, in particular from 60.0 μm to 90.0 μm.

5. The cosmetic product according to any one of the preceding claims, characterized in that the at least one second polymer layer (P2) is formed from polyethylene terephthalate or polyethylene naphthalate, in particular from polyethylene terephthalate.

6. The cosmetic product according to any one of the preceding claims, characterized in that the at least one second polymer layer (P2) has a layer thickness of from 1.00 μm to 100 μm, preferably from 2.50 μm to 50.0 μm, preferably from 5.00 μm to 25.0 μm, in particular from 10.0 μm to 20.0 μm.

7. The cosmetic product according to any one of the preceding claims, characterized in that the at least one barrier layer (BS) is formed from aluminum oxides, magnesium oxides, silicon oxides, titanium oxides, tin oxides, zirconium oxides, inorganic-organic hybrid polymers (ORMOCER polymers) or mixtures thereof, in particular from silicon oxides.

8. The cosmetic product according to any one of the preceding claims, characterized in that the at least one barrier layer (BS) has a layer thickness of from 1.00 nm to 1000 nm, preferably from 5.00 nm to 500 nm, preferably from 10.0 nm to 250 nm, in particular from 10.0 nm to 150 nm.

9. The cosmetic product according to any one of the preceding claims, characterized in that the cosmetic composition (KM) contains the at least one oxidizing compound, in particular hydrogen peroxide, in a total amount of from 0.5 to 20% by weight, preferably from 1.0 to 18% by weight, preferably from 1.2 to 16% by weight, in particular from 1.5 to 15% by weight, in relation to the total weight of the cosmetic composition (KM).

10. The cosmetic product according to any one of the preceding claims, characterized in that the cosmetic composition (KM) contains the at least one thickener, selected from the group of xanthans, in a total amount of from 0.1 to 10% by weight, preferably from 0.2 to 8.0% by weight, preferably from 0.2 to 7.0% by weight, in particular from 0.2 to 6.0% by weight, in relation to the total weight of the cosmetic composition (KM).
